(19) United States
(12) Patent Application Publication
(10) Pub. No.: US 2025/0041165 A1
(43) Pub. Date: Feb. 6, 2025

(54) Multi-Layer Structure

(71) Applicant: Vita Zahnfabrik H. Rauter GmbH & Co. KG, Bad Säckingen (DE)

(72) Inventors: Michael Gödiker, Bad Säkingen (DE); Christopher Neun, Bad Säkingen (DE)

(21) Appl. No.: 18/719,412

(22) PCT Filed: Dec. 16, 2022
(86) PCT No.: PCT/EP2022/086277

§ 371 Date: Jun. 13, 2024

(30) Foreign Application Priority Data
Dec. 23, 2021 (EP) 21217466.8

(51) Int. Cl. A61K 6/818; A61K 6/822; A61K 6/824; C04B 35/486; C04B 35/638

(52) U.S. Cl. CPC A61K 6/818; A61K 6/822; A61K 6/824; C04B 35/486; C04B 35/638; C04B 2235/3217; C04B 2235/3225; C04B 2235/3275; C04B 2235/5436; C04B 2235/608; C04B 2235/6562; C04B 2235/6567; C04B 2235/75; C04B 2235/96; C04B 2235/9661; C04B 2237/348; C04B 2237/58

(57) ABSTRACT

The present invention relates to a molded body with a color profile for use in the production of dental restorations obtainable by sintering a press-molded body comprising two or more color-different ceramic powder layers, wherein each powder layer comprises at least 80 wt. % of ZrO₂ and is essentially free of iron oxide, characterized in that at least one powder layer comprises terbium oxide (Tb₄O₇).

The present invention relates to a sintered molding with a color gradient for use in the preparation of dental restorations, obtainable by sintering a compression molding comprising 2 or more ceramic powder layers having different colors, and a process for the preparation thereof, and the use of the molding for the preparation of dental restorations.

DE 102016106370 A1 describes a process for producing a zirconia-containing colored blank for the preparation of a dental restoration, wherein powdery starting raw materials, of which at least some contain coloring elements, are mixed together, die mixture obtained is compressed and then subjected to a heat treatment. The powder mixtures employed contain elements that produce fluorescence effects, such as bismuth, which may, however, have disadvantageous effects on the sintering performance.

WO 2018/115529 A1 discloses multilayered oxide ceramic bodies, which comprise at least two layers, wherein at least one layer contains lanthanum oxide, and the at least two different layers differ in their lanthanum oxide content. The lanthanum oxide is employed for adapting the sinter performance.

EP 3108849 A1 describes a porous multi-layered and colored zirconia-based blank, wherein the individual powder layers may have a different thickness.

EP 3772497 A1 describes a sintered molding with a color gradient for use in dental restorations that have powder layers, which contain iron oxide, however.

Because of their hardness and good workability as well as because of their controllable translucency properties, zirconia ceramics have entered dental engineering for the production of dental restorations. The coloring of the ZrO₂ ceramics is usually effected by adding coloring oxides, which are sintered together with the zirconia. In order to obtain a gradient of the color that is matched to the natural color of the teeth, changing the concentration of the coloring oxides from layer to layer and adjusting it so that the color gradient gets as close as possible to the natural color of the teeth suggests itself. However, there is a problem in that the coloring metal oxides may have an influence on the sintering performance of the zirconia ceramics. This has been found, in particular, when iron oxides were employed. A layerwise structure of a zirconia ceramic in which the concentration of iron oxide is changed from layer to layer leads to a sintering distortion of the ceramic. Subsequently, such distortion has to be compensated/corrected, which is difficult. Further, a sintering performance that is different from layer to layer also results in a different hardness of the individual layers, which additionally complicates the processing with subtractive means for the preparation of dental restorations.

Therefore, it has been the object of the present invention to provide a sintered molding that solves the above mentioned problems, in particular, that can be sintered without distortion over a large temperature range.

In addition, an object of the present invention has been to provide a ceramic sintered molding with a color gradient that has a substantially homogeneous hardness and thus is well workable.

The above mentioned objects are achieved by a sintered molding according to claim 1 of the present invention.

In a first embodiment, the present invention relates to a sintered molding with a color gradient for use in the preparation of dental restorations, obtainable by sintering a compression molding comprising 2 or more ceramic powder layers having different colors, wherein each powder layer has at least 80% by weight ZrO₂ and is essentially free of iron oxide, characterized in that at least one powder layer includes terbium oxide.

As used in the present invention, “essentially free of iron oxide” means that less than 0.01% by weight, preferably less than 0.001% by weight, or less than 0.0001% by weight, thereof is contained, respectively based on the total weight of the powder layer. In a preferred embodiment, the sintered molding is free of iron oxide.

The use of terbium oxide has proven excellent for both the coloring and the distortion stability.

“Terbium oxide” within the meaning of the present invention basically includes all oxides of terbium. Preferably, said terbium oxide is selected from the group consisting of Tb₂O₃, Tb₄O₇, Tb₇O₁₂, Tb₁₁O₂₀, TbO₂, and mixtures thereof.

More preferably employed can be terbium oxide selected from the group consisting of Tb₄O₇, Tb₇O₁₂, Tb₁₁O₂₀, and any mixtures thereof.

It is especially preferred for the terbium oxide to be in the form of Tb₄O₇, or as a mixture of Tb₄O₇ with Tb₇O₁₂, and/or Tb₁₁O₂₀.

In addition, terbium oxides having a mixed valency are preferred.

In a preferred embodiment, at least one powder layer, preferably at least 2 powder layers, or at least 3 powder layers, or at least 4 powder layers, specifically 5 powder layers, or every powder layer, comprise(s) terbium oxide in an amount of from 0.001 to 0.15% by weight, preferably from 0.01 to 0.1% by weight, especially from 0.02 to 0.08% by weight, specifically from 0.02 to 0.06% by weight, of terbium oxide.

Surprisingly, it has been found that each of the sintered powder layers is subject to an essentially equal volume change over a temperature range of from 25 to 1600° C., especially over a temperature range of from 50 to 1400° C., or from 900 to 1400° C., and specifically over a temperature range of from 900 to 1350° C.

As used in the present invention, an “essentially equal volume change” means that the difference of the volume change at a defined temperature in a range of from 25 to 1600° C. of 2 layers of the blank sintered according to the invention is at most 1%, preferably at most 0.5%, especially at most 0.05%.

This results in a low sintering distortion for the moldings sintered according to the invention.

In a further preferred embodiment of the present invention, each powder layer has a different concentration of terbium oxide, erbium oxide, and cobalt oxide.

In a preferred embodiment, each powder layer exclusively has terbium oxide, erbium oxide, and cobalt oxide as coloring metal oxides.

In another embodiment, each powder layer contains terbium oxide, erbium oxide, and cobalt oxide, but is essentially free of other coloring metal oxides.

As used in the present invention, “essentially free of other coloring metal oxides” means that less than 0.01% by weight, preferably less than 0.001% by weight, or less than 0.0001% by weight, thereof is contained, respectively based on the total weight of the powder layer. In a preferred embodiment, the sintered molding is free of other coloring metal oxides.

In a further preferred embodiment of the present invention, at least one powder layer, preferably 2 or more powder layers, specifically all powder layers, are essentially free of elements that produce fluorescence effects, especially essentially free of bismuth.

As used in the present invention, “essentially free of elements that produce fluorescence effects” means that less than 0.01% by weight, preferably less than 0.001% by weight, or less than 0.0001% by weight, thereof is contained, respectively based on the total weight of the powder layer. In a preferred embodiment, the sintered molding is free of other metal oxides that produce fluorescence effects.

The use of lanthanum oxide in dental restorations may have a disadvantageous effect on the color composition, i.e., the interplay of the coloring metal oxides to be employed in the powder compositions to be employed according to the invention.

Therefore, in a further preferred embodiment of the present invention, at least one powder layer, preferably 2 or more powder layers, specifically all powder layers, are essentially free of lanthanum oxide.

As used in the present invention, “essentially free of lanthanum oxide” means that less than 0.004% by weight, preferably less than 0.001% by weight, or less than 0.0001% by weight, thereof is contained, respectively based on the total weight of the powder layer. In a preferred embodiment, the sintered molding is free of lanthanum oxide.

EP 3108849 A1 discloses a porous, colored and multilayered zirconia blank, in which the layer thickness is set up differently.

Preferably, at least one powder layer, more preferably 2 or more powder layers, especially each powder layer, contain(s) erbium oxide (Er₂O₃) in an amount of from 0.1 to 1.0% by weight, especially from 0.2 to 0.8% by weight, respectively based on the total weight of the powder layer composition. Surprisingly, it has been found that erbium oxide can be employed as a coloring metal oxide in such amounts without adversely affecting the sintering properties.

In a further preferred embodiment of the present invention, the weight ratio of terbium oxide to cobalt oxide is within a range of from 80:1 to 5:1, preferably from 75:1 to 10:1, and specifically from 60:1 to 15:1 in at least one powder layer, preferably in at least 2 or 3 powder layers, especially in each powder layer. In particular, good coloring and sintering properties could be achieved with the above mentioned adjusted weight ratios, especially when Tb₄O₇ is used.

Each powder layer may preferably be prepared by mixing base powders. In a preferred embodiment, the preparation of each powder layer is effected by mixing 2 or more, preferably 3 or more, especially 4 or more, base powders. The use of a limited amount of base powders in different amounts for the preparation of each powder layer has advantages in terms of compatibility of the individual layers stacked on one another, and of their color gradient in the sintered molding. Suitable base powders may be acquired from the company Tosoh, for example.

The sintered moldings of the present invention preferably have a gradual color gradient. In one embodiment of the invention, the blank has layers with different colors, and the sintering process yields a gradual color gradient.

In a preferred embodiment of the present invention, at least 2, preferably all, powder layers have an essentially identical yttria content.

As used in the present invention, an “essentially identical yttria content in 2 or more layers” means that the difference within the layers is not more than 0.1 mole %, preferably not more than 0.05 mole %, especially less than 0.01 mole %.

In an alternative embodiment of the present invention, the sintered molding has a translucency gradient. Preferably, the molding according to the invention exhibits an increase from layer to layer of the yttria content.

In a preferred embodiment, the moldings according to the invention have powder layers each with at least 0.02% by weight Al₂O₃.

It has proven advantageous that each of the powder layers have at least 0.02% by weight Al₂O₃, preferably Al₂O₃ in an amount of from 0.03 to 0.15% by weight, especially Al₂O₃ in an amount of from 0.03 to 0.08% by weight. The preferred amount of Al₂O₃ in the individual powder layers leads to an improved stability and strength performance of the moldings according to the invention.

In another aspect of the present invention, at least one, preferably at least 2, or at least 3, of the powder layers, especially all powder layers, include Y₂O₃ and/or Er₂O₃, preferably in an amount of at least 3% by weight, especially at least 5% by weight, or at least 6% by weight, and especially from 4.5 to 12% by weight, especially from 6 to 10% by weight, based on the total weight of the components of the powder layer.

The Y₂O₃ has a function of stabilizing the zirconia crystal phases and is not a coloring metal oxide within the meaning of the present invention.

The erbium oxide (Er₂O₃) has a function of stabilizing the ZrO₂ crystal phases and is a coloring metal oxide within the meaning of the present invention.

Preferably, the powder layers include zirconia and/or HfO₂ in an amount of at least 89% by weight, preferably in an amount of from 89 to 98% by weight, especially from 90 to 96% by weight, respectively based on the total weight of the components of the powder layer.

In a preferred embodiment, the powder layers comprise zirconia and hafnia, more preferably, the amount of hafnia is from 0.1 to 5% by weight, especially from 0.5 to 2.5% by weight, based on the total weight of zirconia and hafnia.

In a preferred embodiment of the present invention, the compressed molding is constituted of 3 or 4 or especially of 5 different ceramic powder layers.

In a further preferred embodiment of the present invention, the compressed molding consists of 5 ceramic powder layers, wherein the first powder layer comprises from 20 to 30%, preferably from 22 to 28%, the second powder layer comprises from 10 to 20%, preferably from 12 to 18%, the third powder layer comprises from 15 to 25%, preferably from 17 to 23%, the fourth powder layer comprises from 10 to 20%, preferably from 12 to 18%, and the fifth powder layer comprises from 20 to 30%, preferably from 22 to 28%, of the total thickness of the stacked powder layers, and provided that the total thickness sums up to 100%.

In one embodiment of the invention, the sintered molding is preliminarily sintered at first and processed by subtractive methods, preferably followed by final sintering in another step.

The sintered moldings of the present invention can be used, in particular, as dental restorations, or for the preparation of dental restorations.

In another embodiment of the present invention, at least one powder layer, preferably all powder layers, additionally include organic components, preferably in an amount of from 3 to 6% by weight, especially in an amount of from 4 to 5% by weight. Suitable organic components include, in particular, binders and pressing additives, which can be easily removed thermally in a binder-removing step. Suitable binders for zirconia sintered powders are known to those skilled in the art. These include, for example, polyvinyl alcohol (PVA).

Preferably, the layer powders have a bulk density of below 1.2 g/cm³.

It has proven advantageous to employ layer powders having an average grain Size D₅₀ of from 35 μm to 85 μm, preferably from 40 μm to 80 μm, and especially from 50 μm to 70 μm, or from 40 to 60 μm. The granular powders are measured in a dry state by laser diffraction using a Cilas granulometer.

Usually, the inorganic components of the base powders, i.e., after removing the organic components, such as binders etc., have a particle size D₅₀ of from 0.1 to 1 μm, preferably from 0.2 μm to 0.8 μm, and especially from 0.2 μm to 0.7 μm, as measured by laser diffraction. It has been found that the particle sizes provide a positive contribution to sintering and, in particular, to the color gradients between the individual powder layers.

The compressed molding to be sintered according to the invention can be obtained by stacking 2 or 3, or especially 4, or five or more ceramic powder layers layer by layer. The stacking of the layers may be performed, for example, in a cylindrical container to form disks. Usually, uniaxial pressing of the powder layers may be effected after each layer application. This can be done, for example, by using a press plunger, which merely causes a preliminary compaction, however. The uniaxial pressing of the layers perpendicular to the layer surface is preferably effected under a pressure of from 10 to 20 MPa, especially from 12 to 15 MPa.

In another preferred embodiment, the pressing of the layerwise stacked ceramic powder layers to form a compressed molding by uniaxial pressing at first, perpendicular to the layer surface, preferably to form a preliminarily compacted compressed molding having a density of below 2.8 g/cm³, preferably having a density within a range of from 2.5 to 2.75 g/cm³, for example, 2.65 g/cm³. The uniaxial preliminary compaction may result in a better and more intimate mixing state and thus in a more uniform transition between the layers.

In another preferred embodiment, the pressing for preparing the compressed molding is performed isostatically, said isostatic pressing preferably being performed subsequently to an uniaxial preliminary compaction, to form a compressed molding having a density of below 3.4 g/cm³, especially with a density of 2.80 to 3.3 g/cm³, specifically with a density of 2.85 to 3.25 g/cm³. Said isostatic pressing is preferably effected after all layers of the compressed molding have been stacked. Suitable pressures for said isostatic pressing are usually within a range of from 500 to 10000 bar, preferably within a range of from 800 to 8000 bar, for example, from 1000 to 7000 bar, or from 1000 to 3000 bar.

The thickness of the individual powder layers of the compressed molding may vary. In a preferred embodiment, at least two of the ceramic powder layers differ in terms of thickness. Preferably, at least two of the ceramic powder layers of the compressed molding have a difference in thickness of at least 5%. Typically, the compressed moldings may be in the form of cylindrical circular disks with diameters within a range of from 50 to 200 mm, for example, 75 to 150 mm. The total thickness of cylindrical disks may be, for example, within a range of from 8 to 40 mm, preferably from 10 to 30 mm, especially from 13 to 25 mm. The dimensions relate to the compressed molding in its unsintered state.

With respect to the color design and the subsequent processing, it has proven advantageous if at least one of the outer ceramic powder layers, preferably both outer ceramic powder layers of the compressed molding, have a larger thickness than a ceramic powder layer that is in between the outer ceramic powder layers. Especially when the ceramic moldings prepared according to the invention are used for the preparation of dental restorations, the layer structure with at least one thicker outer layer as described above has proven advantageous, because this is a suitable structure for processing in CAD/CAM systems or other subtractive processing methods.

In a particularly preferred embodiment of the present invention, the compressed molding includes five ceramic powder layers, wherein the first powder layer comprises from 20 to 30%, preferably from 22 to 28%, the second powder layer comprises from 10 to 20%, preferably from 12 to 18%, the third powder layer comprises from 15 to 25%, preferably from 17 to 23%, the fourth powder layer comprises from 10 to 20%, preferably from 12 to 18%, and the fifth powder layer comprises from 20 to 30%, preferably from 22 to 28%, of the total thickness of the stacked powder layers, and provided that the total thickness sums up to 100%.

In another embodiment of the present invention, the sintering is effected at a temperature within a range of from 950 to 1100° C., preferably from 980 to 1050° C., to form a presintered ceramic molding (white body). Usually, the sintering is performed over a sufficient period of time for the existing binders to be removed, and for the compressed molding to be provided with sufficient strength for processing by subtractive methods. The presintered and binder-removed compressed moldings are referred to as “white bodies”.

In one embodiment, the sintering to form the white body is performed over a period of more than 30 minutes, preferably more than 1 hour, especially more than 20 hours, or more than 50 hours, for example, from 60 to 200 hours, or from 70 to 150 hours.

In particular, for preparing ceramic dental restorations, it is appropriate that the presintered ceramic molding is processed by subtractive methods, preferably followed by final sintering in another step. When subtractive methods are applied, sinter shrinkage is usually taken into account in the calculations.

Final sintering is usually performed at temperatures above 1350° C., preferably above 1400° C., especially within a range of from 1420° C. to 1600° C., or from 1450° C. to 1590° C., or from 1480° C. to 1580° C.

The sintering time for final sintering is usually a period of more than 4 minutes, preferably more than 5 minutes, especially within a range of from 5 to 120 minutes.

The moldings according to the invention can be employed, in particular, in the dental field. They are characterized by a high edge strength in dental restorations, an excellent structure, and a high three-point bending strength. Therefore, the ceramic moldings of the present invention are preferably dental restorations, such as inlays, onlays, crowns, bridges, or veneers.

The present invention further relates to the use of the ceramic molding according to the invention for dental restorations, or for preparing dental restorations.

The present invention further relates to the use of a base powder comprising zirconium oxide and terbium oxide, especially Tb₄O₇, and yttrium oxide for preparing ceramic dental restorations without sintering distortion, preferably with a color gradient.

The base powder preferably includes yttria in an amount of from 5 to 8% by weight, preferably from 6 to 7% by weight, terbium oxide in an amount of from 0.1 to 0.4% by weight, preferably from 0.1 to 0.3% by weight, and zirconia in an amount of above 80% by weight, preferably from 82% by weight to 94% by weight, especially from 84% by weight to 90% by weight, respectively based on the total weight of the base powder.

The present invention further relates to a process for preparing the sintered molding according to the invention with a color gradient, comprising the following steps:
a) providing 2 or 3 or 4 or 5 or more ceramic powder layers, which are stacked on one another,
b) pressing the layerwise stacked ceramic powder layers to form a compression molding, and
c) sintering the molding obtained in step b) to form a ceramic molding, wherein the ceramic powder layers respectively have different compositions, and wherein each ceramic powder layer includes a mixture of at least two different base powders, and said base powders each have at least 80% by weight ZrO₂, the indicated weight being based on the total weight of the base powder.

Preferred embodiments of the process according to the invention have been explained above.

The present invention further relates to a sintered molding with a layer structure and color gradient for use in the preparation of dental restorations, wherein the molding includes at least three different ceramic powder layers, and each layer consists of at least three or four different base powders, wherein each base powder includes at least 80% by weight ceramic oxides, the indicated weights being respectively based on the total weight of the base powder.

Preferably, the ceramic powder layers include ceramic oxides as defined above. The base powders to be employed respectively correspond to the base powders as defined above.

### EXAMPLES

Table 1 shows 5 base powders A to E that are employed for the compositions of the ceramic powder layers. The grain size D₅₀ of the base powders is within a range of from 40 to 80 μm. The inorganic components of the base powders have a particle size D₅₀ of from 0.2 to 0.7 μm.

The indicated weights are respectively based on the total weight of the powder composition.

TABLE 1

| | | Proportion |
|---|---|---|
| Designation | Component | (% by weight) |
| Base powder A | Y₂O₃ | 6.92 |
| | Al₂O₃ | 0.05 |
| | Organic binder | 4.18 |
| | ZrO₂ | ad 100 |
| Base powder B | Y₂O₃ | 6.86 |
| | Al₂O₃ | 0.05 |
| | Terbium oxide (Tb₄O₇) | 0.20 |
| | Organic binder | 4.26 |
| | ZrO₂ | ad 100 |
| Base powder C | Er₂O₃ | 11.6 |
| | Al₂O₃ | 0.05 |
| | Organic binder | 3.78 |
| | ZrO₂ | ad 100 |
| Base powder D | Y₂O₃ | 6.91 |
| | Al₂O₃ | 0.05 |
| | Co₃O₄ | 0.04 |
| | Organic binder | 4.25 |
| | ZrO₂ | ad 100 |
| Base powder E | Y₂O₃ | 9.92 |
| | Al₂O₃ | 0.06 |
| | Organic binder | 3.7 |
| | ZrO₂ | ad 100 |

The arrangements of the layers set forth in the following Table 2 show the composition of each individual ceramic powder layer in the compressed molding. The compressed moldings are provided for use in the preparation of dental restorations, so that the layer compositions are designed in accordance with the position in the tooth. The compositions of the powder layers are formed from the base powders by varying the proportions to obtain an ideal color gradient. The composition of each powder layer is achieved by homogeneously mixing the base powders in the stated quantities. Subsequently the powders are placed layer by layer into a cylindrical mold having a diameter of 100 mm, and a layer thickness of 18 mm was set. The powder layers are precompressed uniaxially under a pressure of 13 MPa perpendicular to the layer surface, and subsequently compressed isostatically under a pressure of 2000 bar.

Subsequently, binder removing occurs at about 1000° C. over a period of about 100 hours. The thus obtained white bodies are milled using CAD/CAM systems into dental restorations.

These presintered and processed white bodies are subsequently subjected to final sintering at 1450° C. over a period of 120 minutes.

TABLE 2

| | | Base | Base | Base | Base |
|---|---|---|---|---|---|
| | | powder A | powder B | powder C | powder D |
| Powder | Region of | (% by | (% by | (% by | (% by |
| layer | restoration | weight) | weight) | weight) | weight) |
| 1 | Cutting edge | 56.9 | 28.4 | 4.2 | 10.5 |
| 2 | Dentin/cutting | 46.9 | 36.5 | 4.7 | 11.9 |
| | edge | | | | |
| 3 | Dentin | 43.9 | 41.3 | 5.2 | 9.6 |
| 4 | Dentin/neck | 36.2 | 47.2 | 5.5 | 11.1 |
| 5 | Neck | 28.8 | 52.9 | 5.8 | 12.5 |

In the present example, the ceramic powder layers are arranged in such a way that layer 1 (cutting edge) comprises 25%, layer 2 (dentin/cutting edge) comprises 15%, layer 3 (dentin) comprises 20%, layer 4 (dentin/neck) comprises 15%, and layer 5 (neck) comprises 25% of the total thickness of the compressed molding.

Surprisingly, it has been found that a sintering distortion could not be observed even at higher temperatures as compared to formulations containing iron oxide that lead to an identical color design. In comparison, a sintering distortion was found in formulations containing iron oxide, known from the prior art. The sintering increases as the iron oxide proportion increases, so that the Vickers hardness in the white body also increases from lighter layers (low proportion of iron oxide) to darker layers (higher proportion of iron oxide). In contrast, the embodiments according to the invention, which are colored with terbium oxide, are free of distortion throughout the layers, and thus the degree of sintering is also homogeneous throughout the layers, which again leads to a homogeneous distribution of the Vickers hardness and thus also to consistent processing properties in CAM processing.

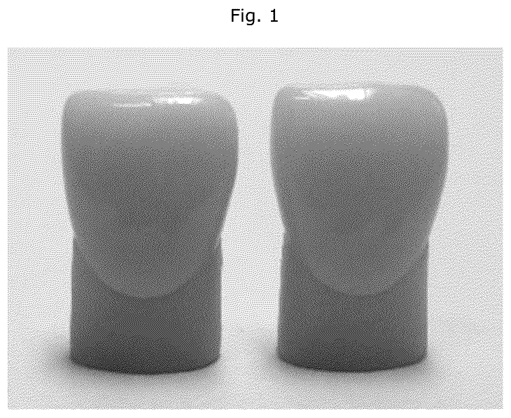

FIG. 1 shows examples of dental restorations obtained from the exemplary ceramic molding. Front tooth crowns 2 are shown.

The layer transitions and color transitions are fluent. The restorations exhibit an excellent edge strength and stability. Reworking and readjusting of the tooth color is not required.

The optimum structure and compositions of the layers shows a shrinkage during sintering that is substantially homogeneous throughout the layers. This is advantageous, in particular, for a perfectly fitting production of the dental restorations, since laborious reworking can be substantially avoided thereby.

The following Table 3 shows examples of different VITA classical A1-D4® tooth colors that can be realized with the base powders.

TABLE 3

| | | Formulation | | |
|---|---|---|---|---|
| | | [% by | | |
| Color | Raw material | weight] | Component | [% by weight] |
| A1 | Base powder A | 84.62 | Y₂O₃ | 6.71459 |
| | Base powder B | 10.07 | Al₂O₃ | 0.04985 |
| | Base powder C | 3.00 | Tb₄O₇ | 0.02193 |
| | Base powder D | 2.31 | Co₃O₄ | 0.00092 |
| | | | Er₂O₃ | 0.34800 |
| | | | Total: | 7.13530 |
| A2 | Base powder A | 78.10 | Y₂O₃ | 6.57312 |
| | Base powder B | 14.00 | Al₂O₃ | 0.04975 |
| | Base powder C | 5.00 | Tb₄O₇ | 0.02972 |
| | Base powder D | 2.90 | Co₃O₄ | 0.00116 |
| | | | Er₂O₃ | 0.5800 |
| | | | Total: | 7.23375 |
| A3 | Base powder A | 70.17 | Y₂O₃ | 6.55361 |
| | Base powder B | 19.44 | Al₂O₃ | 0.04974 |
| | Base powder C | 5.22 | Tb₄O₇ | 0.04049 |
| | Base powder D | 5.17 | Co₃O₄ | 0.00207 |
| | | | Er₂O₃ | 0.60552 |
| | | | Total: | 7.25143 |
| A3.5 | Base powder A | 56.28 | Y₂O₃ | 6.43698 |
| | Base powder B | 28.80 | Al₂O₃ | 0.04966 |
| | Base powder C | 6.80 | Tb₄O₇ | 0.05902 |
| | Base powder D | 8.12 | Co₃O₄ | 0.00325 |
| | | | Er₂O₃ | 0.78880 |
| | | | Total: | 7.33771 |
| A4 | Base powder A | 49.74 | Y₂O₃ | 6.44178 |
| | Base powder B | 30.40 | Al₂O₃ | 0.04967 |
| | Base powder C | 6.70 | Tb₄O₇ | 0.06219 |
| | Base powder D | 13.16 | Co₃O₄ | 0.00526 |
| | | | Er₂O₃ | 0.77720 |
| | | | Total: | 7.33610 |

With respect to the components, the stated weight amounts are supplemented to 100% by weight by the binder and the zirconia.

TABLE 4
Different tooth colors that can be obtained by mixing base
powders. They may serve, for example, as powder layer materials
for the sintered moldings according to the invention.

| | GR A3 | GR A3 | GR A3 | GR A3 | GR A3 |
|---|---|---|---|---|---|
| Granules | A01 | B01 | C01 | D01 | E01 |
| Base powder A [g] | 29.969 | 21.021 | 42.104 | 29.787 | 43.693 |
| Base powder B [g] | 5.349 | 5.160 | 11.664 | 10.004 | 17.416 |
| Base powder C [g] | 1.680 | 1.395 | 3.132 | 2.480 | 4.039 |
| Base powder D [g] | 3.002 | 2.424 | 3.100 | 2.729 | 4.852 |
| Total amount [g] | 40.000 | 30.000 | 60.000 | 45.000 | 70.000 |

TABLE 5
Examples of single layer compositions The powder layer
compositions are obtained by mixing the base powders.

| Granules | GR A3 A02 | GR A3 B02 |
|---|---|---|
| Base powder E [g] | 29.969 | 21.021 |
| Base powder B [g] | 5.349 | 5.160 |
| Base powder C [g] | 1.680 | 1.395 |
| Base powder D [g] | 3.002 | 2.424 |
| Total amount [g] | 40.000 | 30.000 |

The powder compositions for the individual powder layers of the sintered molding according to the invention are obtained by mixing the base powders.

For preparing the multilayer sintered molding, the layer powder compositions listed in Tables 4 and 5 are compressed in a Weber press (program No. 22; 100 MPa=80 kN). The total charge per block weighed 40 g. The different layers are distributed in the following way as set forth in Tables 6 and 7:

TABLE 6

| Powder | | | |
|---|---|---|---|
| composition | Layer | Height [%] | Weighed charge [g] |
| GR A3 A01 | A | 25 | 10 |
| GR A3 B01 | B | 15 | 6 |
| GR A3 C01 | C | 20 | 8 |
| GR A3 D01 | D | 15 | 6 |
| GR A3 E01 | E | 25 | 10 |

TABLE 7

| Powder | | | |
|---|---|---|---|
| composition | Layer | Height [%] | Weighed charge [g] |
| GR A3 A02 | A | 25 | 10 |
| GR A3 B02 | B | 15 | 6 |
| GR A3 C01 | C | 20 | 8 |
| GR A3 D01 | D | 15 | 6 |
| GR A3 E01 | E | 25 | 10 |

The green body density of the pressed blanks is 3.08 g/cm³.

The pressed blanks prepared in Tables 6 and 7 are subsequently freed from binder. The removal of the binder is performed in a Thermo-STAR oven. The removal of the binder or sinter-bonding of the blocks is effected with the following binder removal program (about 20 hours). Tmax=1040° C. (see Table 8).

TABLE 8

| Temperature | Heating rate | Holding time |
|---|---|---|
| [° C.] | [° C./min] | [min] |
| 30 → 280 | 0.7 | — |
| 280 | — | 90 |
| 280 → 400 | 1 | — |
| 400 → 600 | 10 | — |
| 600 → 1040 | 5 | — |
| 1040 | — | 120 |
| 1040 → 30 | 10 | — |

The white body density of the green bodies with removed binder is 3.17 g/cm³.

The Vickers hardness of the white bodies was determined with a Zwick hardness testing machine. Thus, the hardness on the top side and the bottom side was determined each by six measurements and averaging. (testing force 19.61 N; loading level HV 2; waiting time at the load point: 20 s):
Vickers hardness HV 2 (top side): 55.3
Vickers hardness HV 2 (bottom side): 55.0

From the white body block, 2 front tooth crowns are milled and subsequently finally sintered, in which the crowns are heated up to 1450° C. at first, then heated at that temperature for 2 hours, and then continuously cooled down to room temperature.

What is surprising in the sintered bodies according to the invention is the Vickers hardness, in particular. While the Vickers hardness at the presintered blank (white body) increases as the Fe₂O₃ content increases in sintered bodies not according to the invention, the Vickers hardness remains unchanged throughout the block with about 55 HV 2 in sintered bodies according to the invention.

Under an aesthetic aspect, the variant with the higher yttrium content (Table 8) in the cutting edge offers a somewhat higher translucency along the cutting edge.

1. A sintered molding with a color gradient for use in the preparation of dental restorations, obtainable by sintering a compression molding comprising 2 or more ceramic powder layers having different colors, wherein each powder layer has at least 80% by weight ZrO₂ and is essentially free of iron oxide, characterized in that each powder layer has a different concentration of terbium oxide, erbium oxide, and cobalt oxide.

2. The sintered molding according to claim 1, characterized in that each of the sintered powder layers is subject to an essentially equal volume change over a temperature range of from 25 to 1600° C.

3. The sintered molding according to claim 1, characterized in that the weight ratio of terbium oxide to cobalt oxide is within a range of from 80:1 to 5:1 in at least one powder layer.

4. The sintered molding according to claim 1, characterized in that the blank has layers with different colors, and the sintering process yields a gradual color gradient.

5. The sintered molding according to claim 1, characterized in that at least 2, powder layers have an essentially identical yttria content.

6. The sintered molding according to one or more of claim 1, characterized in that said blank has a translucency gradient.

7. The sintered molding according to claim 1, characterized in that each of the powder layers have at least 0.02% by weight Al₂O₃.

8. The sintered molding according to claim 1, characterized in that at least one of the powder layers include Y₂O₃ and/or Er₂O₃ in an amount of at least 3% by weight based on the total weight of the components of the powder layer.

9. The sintered molding according to claim 1, characterized in that the powder layers include zirconia and/or HfO₂ in an amount of at least 89% by weight, based on the total weight of the components of the base powder.

10. The sintered molding according to claim 1, characterized in that the compressed molding consists of 4 or more powder layers.

11. The sintered molding according to claim 1, characterized in that the compressed molding consists of 5 ceramic powder layers, wherein the first powder layer comprises from 20 to 30%, the second powder layer comprises from 10 to 20%, the third powder layer comprises from 15 to 25%, the fourth powder layer comprises from 10 to 20%, and the fifth powder layer comprises from 20 to 30% of the total thickness of the stacked powder layers, and provided that the total thickness sums up to 100%.

12. The sintered molding according to claim 1, characterized in that a presintered ceramic molding is processed by subtractive methods.

13. The sintered molding according to claim 1, characterized in that the molding exhibits an increase from layer to layer of the terbium oxide content.

14. The sintered molding according to claim 1, characterized in that the molding contains terbium oxide in an amount of from 0.001 to 0.15% by weight of terbium oxide in each powder layer.

15. A dental restoration made from the molding according to claim 1.

16. A process for preparing a sintered molding with a color gradient according to claim 1, comprising the following steps:
a) providing 2 or 3 or 4 or 5 or more ceramic powder layers having different colors, which are stacked on one another,
b) pressing the layerwise stacked ceramic powder layers to form a compression molding, and
c) sintering the molding obtained in step b) to form a ceramic molding, wherein the ceramic powder layers respectively have different compositions, and wherein each powder layer has at least 80% by weight ZrO₂ and is essentially free of iron oxide, characterized in that each powder layer has a different concentration of terbium oxide, erbium oxide, and cobalt oxide.

17. The sintered molding according to claim 2, characterized in that each of the sintered powder layers is subject to an essentially equal volume change over a temperature range of from 900 to 1400° C.

18. The sintered molding according to claim 3, characterized in that the weight ratio of terbium oxide to cobalt oxide is within a range of from 75:1 to 10:1 in at least 2 or 3 powder layers.

19. The sintered molding according to claim 1, characterized in that said blank has an increase of the amount of yttrium oxide from layer to layer.

20. The sintered molding according to claim 1, characterized in that each of the powder layers Al₂O₃ is present in an amount of from 0.03% to 0.15% by weight.
